(19) United States
(12) Patent Application Publication
(10) Pub. No.: US 2009/0171178 A1
(43) Pub. Date: Jul. 2, 2009

(54) Method and System for Powering an Electronic Device

(75) Inventors: Lei He, Moraga, CA (US); Erwin S. Budiman, Fremont, CA (US); Kenneth J. Doniger, Menlo Park, CA (US)

Correspondence Address: JACKSON & CO., LLP, 6114 LA SALLE AVENUE, #507, OAKLAND, CA, 94611-2802, US

(73) Assignee: Abbott Diabetes Care, Inc., Alameda, CA (US)

(21) Appl. No.: 11/537,984

(22) Filed: Oct. 2, 2006

Related U.S. Application Data
(63) Continuation-in-part of application No. 11/396,135, filed on Mar. 31, 2006.

(51) Int. Cl. A61B 5/145

(52) U.S. Cl. 600/365

(57) ABSTRACT

Methods and apparatuses for providing power supply to a device are provided.

### RELATED APPLICATION

This application claimed priority to pending application Ser. No. 11/396,135 filed Mar. 31, 2006, entitled “Method and System for Powering an Electronic Device” the disclosure of which is incorporated by reference in its entirely for all purposes

### BACKGROUND

Analyte, e.g., glucose monitoring systems including continuous and discrete monitoring systems generally include a battery powered and microprocessor controlled system which is configured to detect signals proportional to the corresponding measured glucose levels using an electrometer, and RF signals to transmit the collected data. One aspect of certain glucose monitoring systems include a transcutaneous or subcutaneous analyte sensor configuration which is, for example, partially mounted on the skin of a subject whose glucose level is to be monitored. The sensor may use a two or three-electrode (work, reference and counter electrodes) configuration driven by a controlled potential (potentiostat) analog circuit connected through a contact system.

The analyte sensor may be configured so that at least a portion thereof is placed under the skin of the patient so as to detect the analyte levels of the patient, and another portion of segment of the analyte sensor that is in communication with the transmitter unit. The transmitter unit is configured to transmit the analyte levels detected by the sensor over a wireless communication link such as an RF (radio frequency) communication link. To transmit signals, the transmitter unit requires a power supply such as a battery. Generally, batteries have a limited life span and require periodic replacement. More specifically, depending on the power consumption of the transmitter unit, the power supply in the transmitter unit may require frequent replacement, or the transmitter unit may require replacement (e.g, disposable power supply such as disposable battery).

In view of the foregoing, it would be desirable to have an approach to provide a power supply for a transmitter unit in a data monitoring and management system.

### SUMMARY OF THE INVENTION

In view of the foregoing, in accordance with the various embodiments of the present invention, there is provide a housing, an analyte sensor disposed in the housing for detecting one or more analyte levels of a patient, and a power management section disposed in the housing, the power management unit including a power storage unit configured to store charge when in a predetermined proximity to a magnetic field.

These and other objects, features and advantages of the present invention will become more fully apparent from the following detailed description of the embodiments, the appended claims and the accompanying drawings.

### BRIEF DESCRIPTION OF THE DRAWINGS

FIG. 1 illustrates a block diagram of a data monitoring and management system for practicing one embodiment of the present invention;

FIG. 2 is a block diagram of the transmitter of the data monitoring and management system shown in FIG. 1 in accordance with one embodiment of the present invention;

FIG. 3 is a block diagram of a magnetic field generator unit of the receiver unit configured for providing inductive power recharge in the data monitoring and management system in accordance with one embodiment of the present invention;

FIG. 4 illustrates the magnetic field radiation unit of the serial resonant tank section of the receiver unit shown in FIG. 3 in accordance with one embodiment of the present invention;

FIG. 5 is a block diagram illustrating the transmitter unit with a rechargeable battery configured for inductive recharging in the data monitoring and management system in accordance with one embodiment of the present invention;

FIG. 6 illustrates the high frequency power transformer of the transmitter unit and the receiver unit including the magnetic field generator unit of the data monitoring and management system in accordance with one embodiment of the present invention;

FIG. 7 illustrates a data monitoring in accordance with another embodiment of the present invention;

FIG. 8 is a block diagram of the implanted sensor unit of the data monitoring system of FIG. 7 in accordance with one embodiment of the present invention;

FIG. 9 is a block diagram of the transmitter unit of the data monitoring system shown in FIG. 7 in accordance with one embodiment of the present invention;

FIG. 10 illustrates the magnetic field generated between the implanted sensor unit and the on-body transmitter unit in accordance with one embodiment of the present invention;

FIG. 11 illustrates a pot type ferrite core of the inductive antenna in accordance with one embodiment of the present invention;

FIG. 12 illustrates the implanted sensor unit in accordance with one embodiment of the present invention;

FIG. 13 illustrates an insertion device for use in the transcutaneous implantation of the implanted sensor unit in accordance with one embodiment of the present invention.

### DETAILED DESCRIPTION

As described in accordance with the various embodiments of the present invention below, there are provided methods and system for inductively recharging a power source such as a rechargeable battery in an electronic device such as a data transmitter unit used in data monitoring and management systems such as, for example, in glucose monitoring and management systems.

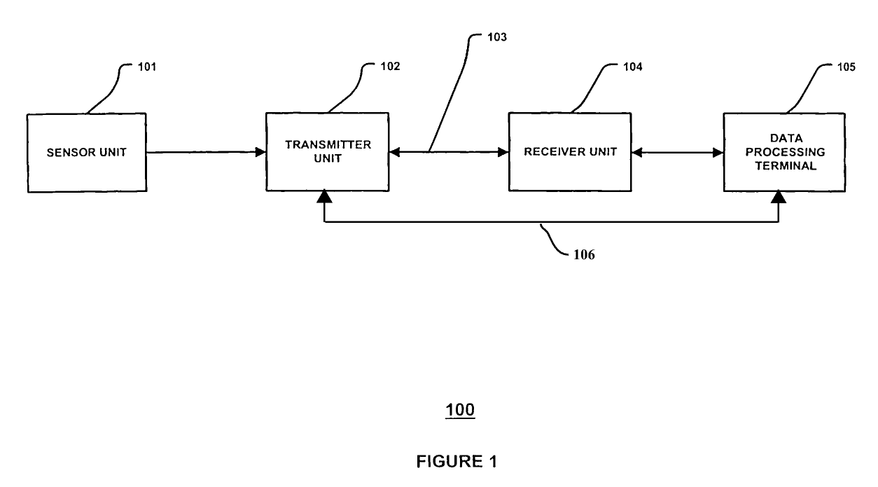

FIG. 1 illustrates a data monitoring and management system such as, for example, an analyte (e.g., glucose) monitoring system 100 in accordance with embodiments of the present invention. The subject invention is further described primarily with respect to a glucose monitoring system for convenience and such description is in no way intended to limit the scope of the invention. It is to be understood that the analyte monitoring system may be configured to monitor a variety of analytes, e.g., lactate, ketones, and the like.

Indeed, analytes that may be monitored include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored.

The embodiment of glucose monitoring system 100 includes a sensor 101, a transmitter 102 coupled to the sensor 101, and a receiver 104 which is configured to communicate with the transmitter 102 via a communication link 103. The receiver 104 may be further configured to transmit data to a data processing terminal 105 for evaluating the data received by the receiver 104. Moreover, the data processing terminal in one embodiment may be configured to receive data directly from the transmitter 102 via a communication link 106 which may optionally be configured for bi-directional communication. In addition, within the scope of the present invention, the receiver 104 may be configured to include the functions of the data processing terminal 105 such that the receiver 104 may be configured to receive the transmitter data as well as to perform the desired and/or necessary data processing to analyze the received data, for example.

Only one sensor 101, transmitter 102, communication link 103, receiver 104, and data processing terminal 105 are shown in the embodiment of the glucose monitoring system 100 illustrated in FIG. 1. However, it will be appreciated by one of ordinary skill in the art that the glucose monitoring system 100 may include one or more sensor 101, transmitter 102, communication link 103, receiver 104, and data processing terminal 105, where each receiver 104 is uniquely synchronized with a respective transmitter 102. Moreover, within the scope of the present invention, the glucose monitoring system 100 may be a continuous monitoring system, or semi-continuous, or a discrete monitoring system.

In one embodiment of the present invention, the sensor 101 is physically positioned in or on the body of a user whose glucose level is being monitored. The sensor 101 may be configured to continuously sample the glucose level of the user and convert the sampled glucose level into a corresponding data signal for transmission by the transmitter 102. In one embodiment, the transmitter 102 is mounted on the sensor 101 so that both devices are positioned on the user's body. The transmitter 102 may perform data processing such as filtering and encoding of data signals, each of which corresponds to a sampled glucose level of the user, for transmission to the receiver 104 via the communication link 103.

In one embodiment, the glucose monitoring system 100 is configured as a one-way RF communication path from the transmitter 102 to the receiver 104. In such embodiment, the transmitter 102 transmits the sampled data signals received from the sensor 101 without acknowledgement from the receiver 104 that the transmitted sampled data signals have been received. For example, the transmitter 102 may be configured to transmit the encoded sampled data signals at a fixed rate (e.g., at one minute intervals) after the completion of the initial power on procedure. Likewise, the receiver 104 may be configured to detect such transmitted encoded sampled data signals at predetermined time intervals. Alternatively, the glucose monitoring system 100 may be configured with a bi-directional RF (or otherwise) communication between the transmitter 102 and the receiver 104.

Additionally, in one aspect, the receiver 104 may include two sections. The first section is an analog interface section that is configured to communicate with the transmitter 102 via the communication link 103. In one embodiment, the analog interface section may include an RF receiver and an antenna for receiving and amplifying the data signals from the transmitter 102, which are thereafter, demodulated with a local oscillator and filtered through a band-pass filter. The second section of the receiver 104 is a data processing section which is configured to process the data signals received from the transmitter 102 such as by performing data decoding, error detection and correction, data clock generation, and data bit recovery.

In operation, the receiver 104 is configured to detect the presence of the transmitter 102 within its range based on, for example, the strength of the detected data signals received from the transmitter 102 or a predetermined transmitter identification information. Upon successful synchronization with the corresponding transmitter 102, the receiver 104 is configured to begin receiving from the transmitter 102 data signals corresponding to the user's detected glucose level. More specifically, the receiver 104 in one embodiment is configured to perform synchronized time hopping with the corresponding synchronized transmitter 102 via the communication link 103 to obtain the user's detected glucose level.

Referring again to FIG. 1, the data processing terminal 105 may include a personal computer, a portable computer such as a laptop or a handheld device (e.g., personal digital assistants (PDAs)), and the like, each of which may be configured for data communication with the receiver via a wired or a wireless connection. Additionally, the data processing terminal 105 may further be connected to a data network (not shown) for storing, retrieving and updating data corresponding to the detected glucose level of the user.

Within the scope of the present invention, the data processing terminal 105 may include an infusion device such as an insulin infusion pump or the like, which may be configured to administer insulin to patients, and which may be configured to communicate with the receiver unit 104 for receiving, among others, the measured glucose level. Alternatively, the receiver unit 104 may be integrated with an infusion device so that the receiver unit 104 is configured to administer insulin therapy to patients, for example, for administering and modifying basal profiles, as well as for determining appropriate boluses for administration based on, among others, the detected glucose levels received from the transmitter 102.

Additionally, the transmitter 102, the receiver 104 and the data processing terminal 105 may each be configured for bidirectional wireless communication such that each of the transmitter 102, the receiver 104 and the data processing terminal 105 may be configured to communicate (that is, transmit data to and receive data from) with each other via the wireless communication link 103. More specifically, the data processing terminal 105 may in one embodiment be configured to receive data directly from the transmitter 102 via the communication link 106, where the communication link 106, as described above, may be configured for bidirectional communication.

In this embodiment, the data processing terminal 105 which may include an insulin pump or the like, may be configured to receive the glucose signals from the transmitter 102, and thus, incorporate the functions of the receiver 104 including data processing for managing the patient's insulin therapy and glucose monitoring. In one embodiment, the communication link 103 may include one or more of an RF communication protocol, an infrared communication protocol, a Bluetooth enabled communication protocol, an 802.11x wireless communication protocol, or an equivalent wireless communication protocol which would allow secure, wireless communication of several units (for example, per HIPPA requirements) while avoiding potential data collision and interference.

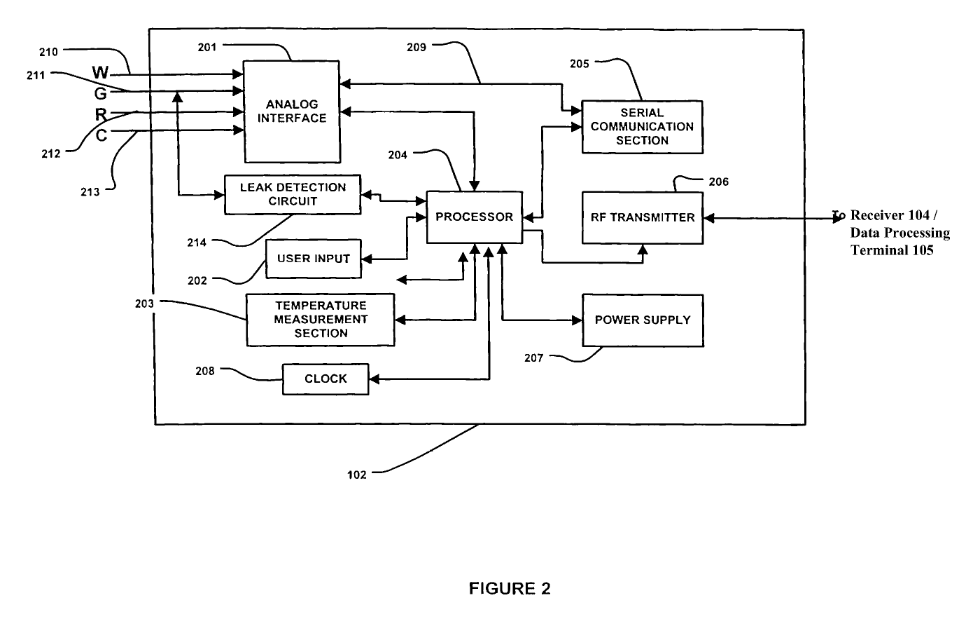

FIG. 2 is a block diagram of the transmitter of the data monitoring and detection system shown in FIG. 1 in accordance with one embodiment of the present invention. Referring to the Figure, the transmitter 102 in one embodiment includes one or more of the following components. The transmitter may include an analog interface 201 configured to communicate with the sensor 101 (FIG. 1), a user input 202, and a temperature detection section 203, each of which is operatively coupled to a transmitter processor 204 such as a central processing unit (CPU). As can be seen from FIG. 2, there are provided four contacts, three of which are electrodes—work electrode (W) 210, guard contact (G) 211, reference electrode (R) 212, and counter electrode (C) 213, each operatively coupled to the analog interface 201 of the transmitter 102 for connection to the sensor unit 201 (FIG. 1). In one embodiment, each of the work electrode (W) 210, guard contact (G) 211, reference electrode (R) 212, and counter electrode (C) 213 may be made using a conductive material that is either printed or etched, for example, such as carbon which may be printed, or metal foil (e.g., gold) which may be etched.

Further shown in FIG. 2 are a transmitter serial communication section 205 and an RF transmitter 206, each of which is also operatively coupled to the transmitter processor 204. Moreover, a power supply 207 such as a battery is also provided in the transmitter 102 to provide the necessary power for the transmitter 102. Additionally, as can be seen from the Figure, clock 208 is provided to, among others, supply real time information to the transmitter processor 204.

In one embodiment, a unidirectional input path is established from the sensor 101 (FIG. 1) and/or manufacturing and testing equipment to the analog interface 201 of the transmitter 102, while a unidirectional output is established from the output of the RF transmitter 206 of the transmitter 102 for transmission to the receiver 104. In this manner, a data path is shown in FIG. 2 between the aforementioned unidirectional input and output via a dedicated link 209 from the analog interface 201 to serial communication section 205, thereafter to the processor 204, and then to the RF transmitter 206. As such, in one embodiment, via the data path described above, the transmitter 102 is configured to transmit to the receiver 104 (FIG. 1), via the communication link 103 (FIG. 1), processed and encoded data signals received from the sensor 101 (FIG. 1). Additionally, the unidirectional communication data path between the analog interface 201 and the RF transmitter 206 discussed above allows for the configuration of the transmitter 102 for operation upon completion of the manufacturing process as well as for direct communication for diagnostic and testing purposes.

As discussed above, the transmitter processor 204 is configured to transmit control signals to the various sections of the transmitter 102 during the operation of the transmitter 102. In one embodiment, the transmitter processor 204 also includes a memory (not shown) for storing data such as the identification information for the transmitter 102, as well as the data signals received from the sensor 101. The stored information may be retrieved and processed for transmission to the receiver 104 under the control of the transmitter processor 204. Furthermore, the power supply 207 may include a commercially available battery.

The power supply section 207 provides power to the transmitter for a minimum amount of time, e.g., about three months of continuous operation after having been stored for a certain period of time, e.g., about eighteen months in a low-power (non-operating) mode. It is to be understood that the described three month power supply and eighteen month low-power mode are exemplary only and are in no way intended to limit the invention as the power supply may be less or more than three months and/or the low power mode may be less or more than eighteen months. In one embodiment, this may be achieved by the transmitter processor 204 operating in low power modes in the non-operating state, for example, drawing no more than approximately 1 μA of current. Indeed, in one embodiment, during the manufacturing process of the transmitter 102 it may be place the transmitter 102 in the lower power, non-operating state (i.e., post-manufacture sleep mode). In this manner, the shelf life of the transmitter 102 may be significantly improved. Moreover, as shown in FIG. 2, while the power supply unit 207 is shown as coupled to the processor 204, and as such, the processor 204 is configured to provide control of the power supply unit 207, it should be noted that within the scope of the present invention, the power supply unit 207 is configured to provide the necessary power to each of the components of the transmitter unit 102 shown in FIG. 2.

Referring back to FIG. 2, the power supply section 207 of the transmitter 102 in one embodiment may include a rechargeable battery unit that may be recharged by a separate power supply recharging unit (for example, provided in the receiver unit 104 or in a mount to which the transmitter may be coupled, e.g., for on-body securement) so that the transmitter 102 may be powered for a longer period of usage time. Moreover, in one embodiment, the transmitter 102 may be configured without a battery in the power supply section 207, in which case the transmitter 102 may be configured to receive power from an external power supply source (for example, a battery) as discussed in further detail below.

Referring yet again to FIG. 2, the temperature detection section 203 of the transmitter 102 is configured to monitor the temperature of the skin near the sensor insertion site. The temperature reading may be used to adjust the glucose readings obtained from the analog interface 201. The RF transmitter 206 of the transmitter 102 may be configured for operation in the frequency band of about 315 MHz to about 470 MHz, for example, in the United States. Further, in one embodiment, the RF transmitter 206 is configured to modulate the carrier frequency by performing Frequency Shift Keying and Manchester encoding. In one embodiment, the data transmission rate is 19,200 symbols per second, with a minimum transmission range for communication with the receiver 104.

Referring yet again to FIG. 2, also shown is a leak detection circuit 214 coupled to the guard electrode (G) 211 and the processor 204 in the transmitter 102 of the data monitoring and management system 100. The leak detection circuit 214 in accordance with one embodiment of the present invention may be configured to detect leakage current in the sensor 101 to determine whether the measured sensor data are corrupt or whether the measured data from the sensor 101 is accurate.

Additional detailed description of the continuous glucose monitoring system, its various components including the functional descriptions of the transmitter are provided in U.S. Pat. No. 6,175,752 issued Jan. 16, 2001 entitled “Analyte Monitoring Device and Methods of Use”, and in application Ser. No. 10/745,878 filed Dec. 26, 2003 entitled “Continuous Glucose Monitoring System and Methods of Use”, and elsewhere.

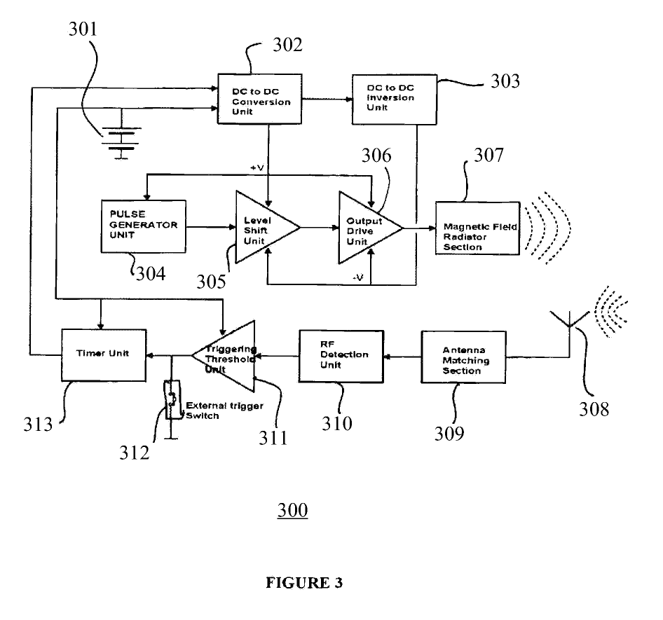

FIG. 3 is a block diagram of a magnetic field generator unit of the receiver unit (or other component) configured for providing inductive power recharge in the data monitoring and management system in accordance with one embodiment of the present invention. Referring to FIG. 3, the magnetic field generator unit 300 includes a power source such as a battery 301 configured to provide DC power to the magnetic field generator unit 300. Also shown in FIG. 3 is a DC to DC conversion unit 302 operatively coupled to the power source 301 and a DC to DC inversion unit 303. The magnetic field generator unit 300 in one embodiment also includes a pulse generator unit 304 operatively coupled to a level shift unit 305 which is in turn, operatively coupled to an output driver unit 306.

Referring again to FIG. 3, the output driver unit 306 is operatively coupled to a magnetic field radiation section 307 which, as described in further detail below, may be configured to generate and radiate a magnetic field. Also shown in FIG. 3 is an RF receiver antenna 308 which is configured to receive data from the transmitter unit 102 (FIG. 1) over the communication link 103 (FIG. 1). Additionally, referring still to FIG. 3, the RF receiver antenna 308 is operatively coupled to an antenna matching section 309 which in turn, is operatively coupled to an RF detection unit 310 which maybe configured to rectify the received RF signal from the transmitter unit 103 as discussed in further detail below. In addition, the RF detection unit 310 as shown in FIG. 3 is operatively coupled to a triggering threshold unit 311. The triggering threshold unit 311 is also operatively coupled to an external trigger switch 312 and a timer unit 313. In one embodiment, the timer unit 313 is operatively coupled to the power source 301 and the DC to DC conversion unit 302, and may be configured to control power supply in the magnetic field generator unit 300 to preserve power consumption and effectively conserve the life of the power source 301.

In one embodiment, the power source 301 is configured to provide direct current (DC) power supply for the magnetic field generator unit 300 that is provided in the receiver unit 104 (FIG. 1) of the data monitoring and management system 100. Alternatively, the magnetic field generator unit 300 may be incorporated into a separate unit or component and used to charge the power supply of the transmitter unit 102.

Referring back to FIG. 3, the DC to DC conversion unit 302 in one embodiment includes a step up DC to DC converter which is configured to boost the voltage level of the power source 301 to a higher positive DC voltage for the pulse generator unit 304, the level shift unit 305, and the output driver unit 306. The DC to DC inversion unit 303 in one embodiment may include a step up DC to DC inverter configured to boost the positive DC voltage received from the DC to DC conversion unit 303 to a negative DC voltage to increase signal swing dynamic range between the positive and negative power supply rails for the level shift unit 305 and the output drive unit 306.

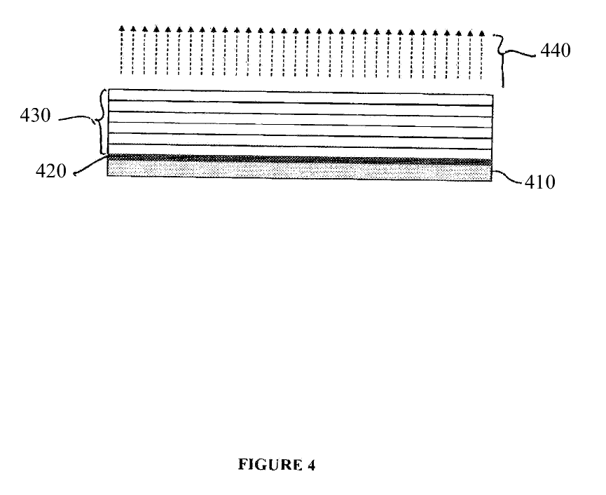

Referring still to FIG. 3, the pulse generator unit 304 in one embodiment includes a square wave generator and configured to generate square wave signals from, for example, approximately 100 KHz to approximately 1 MHz and to provide the generated square wave signals to the level shift unit 305. The frequency range specified above may vary depending upon the specific component used and other design considerations. With the received square wave signals, the level shift unit 305 in one embodiment is configured to convert the positive square wave signals into corresponding positive and negative swing square wave signals with doubled voltage amplitude, which is provided to the output drive unit 306. The output drive unit 306, in turn, is configured to drive the magnetic field radiation section 307 by applying the full swing square wave signals from the level shift unit 305. In one embodiment, as discussed in further detail below in conjunction with FIG. 4, the magnetic field radiation section 307 includes a serial inductor-capacitor (LC) resonance circuit that may includes tuning capacitors and multilayered PCB core coil inductor.

Referring yet again to FIG. 3, the RF receiver antenna 308 in one embodiment is configured to receive the RF signals from the transmitter unit 102 (which may be associated with monitored or detected analyte levels received from the sensor unit 101 (FIG. 1)). In one embodiment, the resonance frequency of the RF receiver antenna 308 may be tuned at the same frequency of the RF carry signal from the transmitter unit 103. The antenna matching circuit 309 is configured to receive the RF signals from the RF receiver antenna 308, and to deliver the received energy from the RF receiver antenna 308 to the RF detection unit 310. In one aspect, the RF detection unit 310 maybe configured to use a zero bias or biased RF Schottkey barrier diode to rectify the amplitude envelope of the received RF signals from the RF receiver antenna 308.

Referring yet still to FIG. 3, the rectified signal from the RF detection unit 310 is provided to the triggering threshold unit 311 which, in one embodiment includes a voltage comparator that compares the signal amplitude level of the rectified signal from the RF detection unit 310 and a reference voltage. Thereafter, the triggering threshold unit 311 in one embodiment is configured to switch the output of the triggering threshold unit 311 to low logical level when the signal level from the RF detection unit 310 exceeds the reference voltage. Similarly, an external trigger switch 312 may be provided which is configured to pull down the output voltage of the triggering threshold unit 311 to a low logical level when the external trigger switch 312 is activated. In one embodiment, the external trigger switch 312 is provided to allow the user to manually turn on the magnetic field generator unit 300.

The triggering threshold unit 311 may be coupled to the timer unit 313 which in one embodiment includes a mono-stable timer, and may be configured to be triggered by the triggering threshold unit 311 to turn on or turn off the magnetic field generator 300 automatically and conserve the battery life of the power source 301. More specifically, in one embodiment, the timer unit 313 may be programmed to a time period that is longer than one time interval between two received RF signals from the transmitter unit 102, but which is shorter than two time intervals, such that the magnetic field generator unit 300 is configured to be turned on continuously when the RF signals are received by the RF receiver antenna 308.

In this manner, in one embodiment of the present invention, the magnetic field generator unit 300 may be configured to inductively charge the rechargeable power source of the transmitter unit 102 (FIG. 1). More specifically, when the transmitter unit 102 is positioned in close proximity to the magnetic field generator unit 300 (for example, incorporated into the receiver unit 104), the magnetic field generator unit 300 may be configured to activate automatically or manually depending upon the transmitter unit 102 transmission status.

That is, in one embodiment, when the transmitter unit 102 is transmitting RF signals, these signals received by the receiver unit 104 including the magnetic field generator unit 300 will activate the magnetic field generator unit 300 as described above by the RF receiver antenna 308 providing the received RF signals to the RF detection unit 310 via the antenna matching section 309. The rectified amplitude envelope signals from the RF detection unit 310 is then configured to pull down the output voltage of the triggering threshold unit 311 to a low logical level. The low logical level starts the mono stable timer unit 313, which turns on the DC to DC conversion unit 302 and for the pulse generator unit 304, the level shift unit 305, and the output drive unit 306 to generate the magnetic field which is then used to inductively recharge the power source in the transmitter unit 102.

In this manner, the RF signal transmission from the transmitter unit 102 in one embodiment is configured to maintain the magnetic field generator unit 300 to continuously generate the magnetic field, or alternatively, the trigger switch 312 may be activated to manually trigger the magnetic field generator unit 300 to continuously generate the magnetic field to inductively recharge the power supply of the transmitter unit 102.

FIG. 4 illustrates the magnetic field radiation section 307 shown in FIG. 3 in accordance with one embodiment of the present invention. Referring to FIG. 4, the magnetic field radiation section 307 of FIG. 3 in one embodiment includes a flexible ferrite layer 410 having disposed thereon an adhesive layer 420 on which, there is provided multilayered PCB core coil inductor 430. In this manner, when the magnetic field generator unit 300 (FIG. 3) is activated, the magnetic field 440 is generated as shown by the directional arrows in FIG. 4. The flexible ferrite layer 410 increases the permeability of the PCB core coil inductor 430 by confining the bottom magnetic field in close proximity to the magnetic field radiation section 307. For a given coil inductor, the inductance is proportional to the permeability of the core material. Furthermore, since Q factor of the inductor is proportional to inductance of the inductor, in one embodiment, the Q factor and inductance of the multilayered PCB core coil inductor 430 are increased by the present of the flexible ferrite layer 410. Moreover, the resonance voltage and current developed on the multilayered PCB core coil inductor 430 is proportional to the Q factor. The magnetic field is, therefore, enhanced.

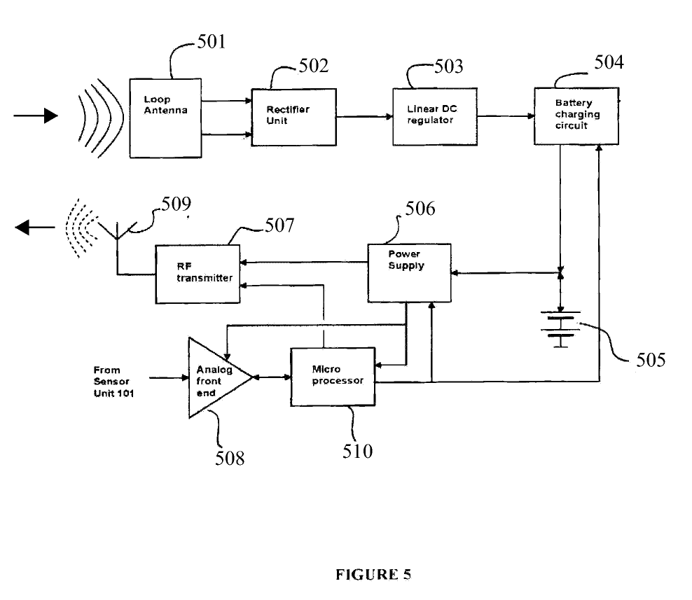

FIG. 5 is a block diagram illustrating the transmitter unit with a rechargeable battery configured for inductive recharging in the data monitoring and management system in accordance with one embodiment of the present invention. Referring to FIG. 5, the transmitter unit 102 with inductive power recharge capability includes an antenna 501 which in one embodiment includes a parallel resonant loop antenna configured to resonate at the same frequency as the magnetic field generated by the magnetic field generator unit 300 (FIG. 3). The generated magnetic field 440 (FIG. 4) induces a current flow in the antenna 501 of the transmitter unit 102 when the transmitter unit 102 is positioned in close proximity to the magnetic field generator unit 300 (for example, when the transmitter unit 102 is placed on top of the magnetic field generator unit 300). The induced current flow then builds up AC voltage across the two ends of the loop antenna 501.

Referring back to FIG. 5, also shown is a rectifier unit 502 which, in one embodiment includes a full bridge rectifier, and is configured to rectify the AC voltage built up in the loop antenna 501 into a corresponding DC voltage. In turn, a linear DC regulator unit 503 is provided to convert the varying DC voltage from the rectifier unit 502 into a constant voltage which is provided to a battery charging circuit 504. The battery charging circuit 504 in one embodiment is configured to provide a constant charging current to charge a rechargeable battery 505 provided in the transmitter unit 102. Accordingly, in one embodiment, the rechargeable battery 505 may be configured to store the energy from the battery charging circuit 504 to provide the necessary power to drive the circuitry and components of the transmitter unit 102.

As shown in FIG. 5, an RF antenna 509 is coupled to an RF transmitter 507 which, under the control of a microprocessor 510 is configured to transmit RF signals that are associated with analyte levels monitored by an sensor unit 101 and processed by an analog front end section 508 which is configured to interface with the electrodes of the sensor unit 101 (FIG. 1). A power supply 506 is optionally provided to provide additional power to the transmitter unit 102.

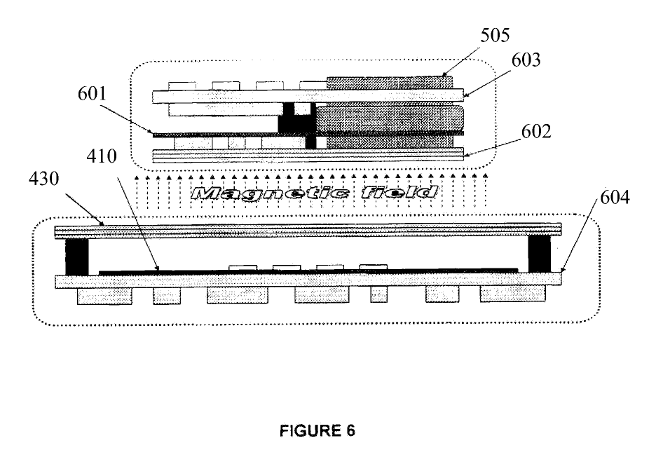

FIG. 6 illustrates the high frequency power transformer of the transmitter unit and the receiver unit including the magnetic field generator unit of the data monitoring and management system in accordance with another embodiment of the present invention. Referring to FIG. 6, as can be seen, a high frequency power transformer is formed by the magnetic field radiation section 307 including the flexible ferrite layer 410 with the multilayered PCB core coil inductor 430 (for example, as similarly shown in FIG. 4), and a similar flexible ferrite layer 601 with a corresponding multilayered PCB core coil inductor 602 provided in the transmitter unit 102. The multilayered PCB core coil inductor 602 in one embodiment includes the loop antenna 501, the rectifier unit 502, and the linear DC regulator unit 503. As shown, when the transmitter unit 102 is positioned in close proximity to the magnetic field generator unit 300 of the receiver unit 104, for example, the high frequency power transformer is generated so as to inductively charge the rechargeable battery 505 of the transmitter unit 102.

Moreover, referring to FIG. 6, the circuit board 603 is configured in one embodiment to include the electronic components associated with the transmitter unit 102, for example, as discussed above in conjunction with FIGS. 2 and 5, while circuit board 604 is configured in one embodiment to include the electronic components associated with the receiver unit 104 including the magnetic field generator section 300. For example, in one embodiment, the circuit board 603 includes the power supply 506, the RF transmitter 507, the analog front end section 508, the RF antenna 509, and the microprocessor 510 as described above in conjunction with FIG. 5.

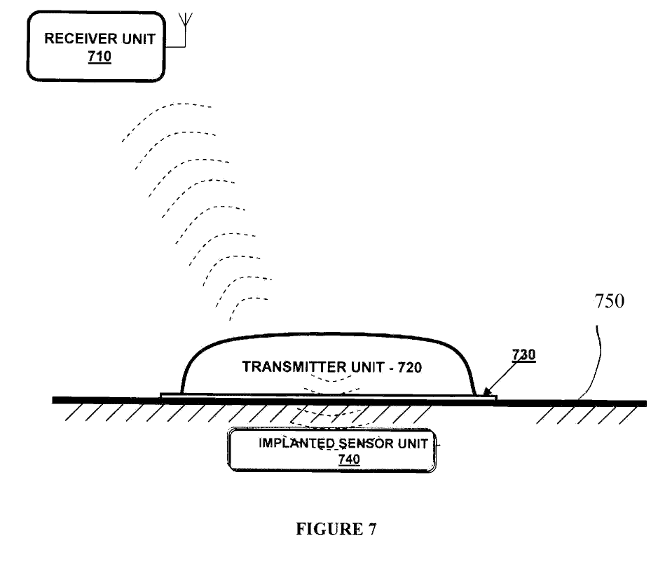

FIG. 7 illustrates a data monitoring in accordance with another embodiment of the present invention. Referring to FIG. 7, in one embodiment, data monitoring system such as analyte monitoring system 700 includes a receiver unit 710 configured to receive one or more data from a transmitter unit 720. As shown, the receiver unit 710 and the transmitter unit 720 may be configured for wireless communication including, for example, RF wireless communication. Within the scope of the present invention, the wireless communication between the transmitter unit 720 and the receiver unit 710 may include bidirectional communication, or alternatively, a uni-directional communication where the transmitter unit 720 is configured to transmit data received from, for example, implanted sensor unit 740, to transmit to the receiver unit 710.

Referring to FIG. 7, in one embodiment, the transmitter unit 720 is provided with an adhesive layer 730 so as to detachably attach to a surface 750, for example, the skin surface of the patient. In one embodiment, the adhesive layer 730 may be configured to securely attach the transmitter unit 720 housing on the skin surface 750 during the usage period of the transmitter unit 720 which may include, for example, approximately, 30 days, 180 days, or a shorter or longer period. Referring again to FIG. 7, the implanted sensor unit 740 in one embodiment is configured to be implanted under the skin layer, for example, of the patient. In one embodiment, the implanted sensor unit 740 may be configured to be surgically implanted with local anesthesia.

In this manner, in one embodiment, the implanted sensor unit 740 may be configured with a magnetically coupled antenna that is configured to transmit data associated with one or more analyte levels of the patient monitored by the implanted sensor unit 740, and further, wherein the implanted sensor unit 740 may be configured to receive power from the on-body transmitter unit 720 via the magnetically coupled antenna. Accordingly, in one embodiment, the implanted sensor unit 740 may be configured to be powered by the magnetic coupling with the transmitter unit 720, and thus may be configured without a separate power supply such as a battery. Accordingly, in one embodiment, a compact, miniaturized size of the implanted sensor unit 740 may be provided.

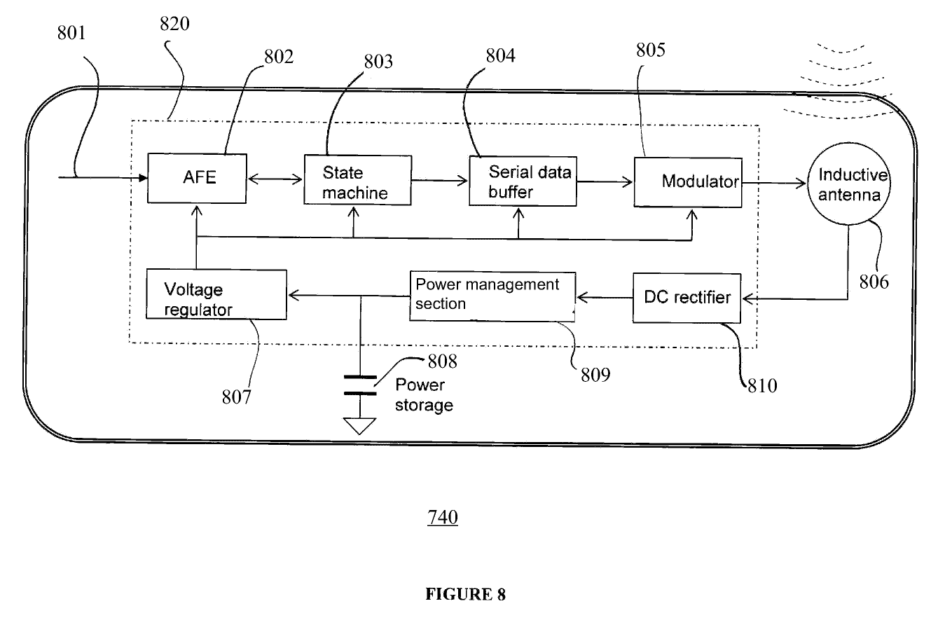

FIG. 8 is a block diagram of the implanted sensor unit of the data monitoring system of FIG. 7 in accordance with one embodiment of the present invention. Referring to FIG. 8, in one embodiment, the implanted sensor unit 740 may include a sensor 801 such as an analyte sensor which is configured to be in fluid contact with an analyte of the patient under the skin layer. In one embodiment, the sensor 801 may be coupled to the analog front end unit (AFE) 802 that may be configured to be in electrical communication with the sensor 801. In one aspect, the AFE unit 802 may be configured to receive one or more signals from the sensor 801 which is associated with a detected or monitored analyte level of the patient.

Referring to FIG. 8, the AFE unit 802 in one embodiment may be configured to perform current to frequency conversion of the received signals from the sensor 801, and thereafter, provide the current to frequency converted signals to a state machine 803. In one embodiment, the state machine 803 may be configured to encode the data received from the AFE unit 802, and thereafter provide the encoded signal to a serial data buffer 804. In one aspect, the state machine may include a logic timer and perform encoding based on, for example, Manchester encoding, Reed Solomon encoding, CRC (cyclic redundancy check) and the like, so as to provide serial data which corresponds to the sensor signals associated with the detected analyte levels to the serial data buffer 804.

The serial data buffer 804 in one embodiment is configured to further process the serial data received from the state machine 803, for example, by performing filtering and the like, and then provide the processed serial signals to a modulator 805. In one embodiment, the modulator 805 may be configured to modulate the processed signals from the serial data buffer, and thereafter provide the modulated signals to an inductive antenna 806 for transmission to the transmitter unit 720 (FIG. 7).

In one aspect, the inductive antenna is configured to change impedance based on the strength of the magnetic field, for example, generated by the transmitter unit 720 (FIG. 7). More specifically, in one embodiment, modulator 805 may include one or more switching circuits operatively coupled to the inductive antenna 806, and where the one or more switching circuits may be configured to tune the inductive antenna 806.

That is, in one embodiment, a serial data value of high (“1”) may be configured to turn off the one or more switching circuits such that the inductive antenna 806 is maintained at the tuning point with maximum impedance. On the other hand, when the serial data value is switched to a low value (“0”), the one or more switching circuits is configured to turn on and to effectively detune the inductive antenna 806 so as to be at a low impedance state. In one embodiment, turning on the one or more switching circuits may effectively short a capacitor coupled to the inductive antenna 806 to the ground terminal.

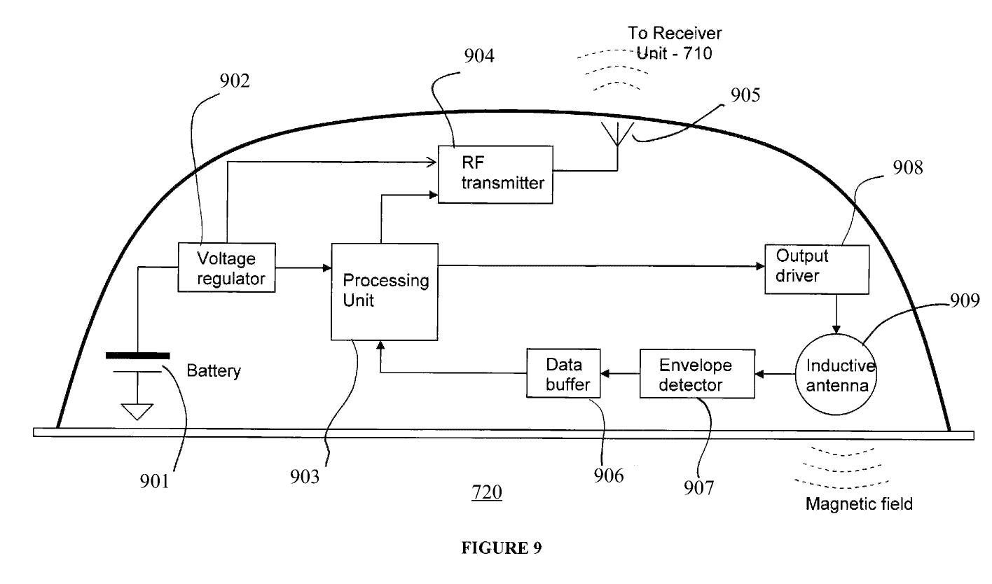

Referring again to FIG. 8, in one embodiment, the inductive antenna 806 may be configured to magnetically couple to a corresponding inductive antenna 909 (FIG. 9) of the transmitter unit 720 that is configured to generate a magnetic field. Accordingly, the inductive antenna 806 of the implanted sensor unit 740 may be configured in one embodiment to magnetically couple with the transmitter unit 720 for powering the internal components of the implanted sensor unit 740, and further to transmit sensor data or signals to the transmitter unit 720. Referring still to FIG. 8, in one embodiment, the implanted sensor unit 740 further includes a DC rectifier 810 that is configured to rectify the signals received from the inductive antenna 806 into corresponding DC signals, and thereafter, provide the rectified DC signals to the power management section 809. In one aspect, the power management section 809 of the implanted sensor unit 740 may be configured to control the power supply to the components of the implanted sensor unit 740, in conjunction with a power storage unit 808, for example, which may include a capacitor, and a voltage regulator 807.

More specifically, in one embodiment, the power management section 809 may be configured to store charge based on the rectified DC signals received from the DC rectifier 810 during the time the magnetic field between the inductive antenna 806 of the implanted sensor unit 710 and the inductive antenna 909 (FIG. 9) of the transmitter unit 720 is above a predetermined strength level. When the magnetic field generated between the transmitter unit 720 and the implanted sensor unit 740 falls below the predetermined strength level, the power management section 809 in one embodiment may be configured to provide power supply to the components of the implanted sensor unit 740 using the charge stored, for example, in the power storage unit 808 which may be configured to store charge during the time period when the magnetic field strength between the transmitter unit 720 and the implanted sensor unit 740 is above the predetermined strength level. Referring still to FIG. 8, the voltage regulator 807 in one embodiment is coupled to each of the AFE unit 802, the state machine 803, the serial data buffer 804 and the modulator 805, and may be configured to regulate the necessary voltage level for each of the components for providing sufficient power to maintain functional operational state.

Referring to FIG. 8, in one embodiment, the AFE unit 802, the state machine 803, the serial data buffer 804, the modulator 805, the voltage regulator 807, power management section 809 and the DC rectifier 810 may be implemented as a single application specific integrated circuit (ASIC) chip.

FIG. 9 is a block diagram of the transmitter unit of the data monitoring system shown in FIG. 7 in accordance with one embodiment of the present invention. Referring to FIG. 9, in one embodiment, the transmitter unit 720 may include a power supply such as a battery 901 that is operatively coupled to a voltage regulator 902 for providing appropriate power signals to the components of the transmitter unit 720. Also shown in FIG. 9 is a processing unit that is operatively coupled to an RF transmitter, an output driver 908, and a data buffer 906. The processing unit 903 which may include one or more microprocessors in one embodiment may be configured to control the data transmission to the receiver unit 710 (FIG. 7) via antenna 905.

Referring back to FIG. 9, the inductive antenna 909 of the transmitter unit 720 may be configured in one embodiment to generate a magnetic field to inductively couple to the implanted sensor unit 740. As discussed above, the inductive antenna 909 in one embodiment may be configured to receive one or more signals from the implanted sensor unit 710 that is associated with the corresponding one or more monitored analyte levels. More specifically, there is provided an envelope detector 907 which in one embodiment is configured to detect a change in the impedance of the inductive antenna 806 (FIG. 8) of the implanted sensor unit 740. In addition, the envelope detector 907 may be further configured to receive signals from the inductive antenna 909 and provide the detected signals to the data buffer 906 which, in one embodiment is configured to demodulate the modulated signals received from the implanted sensor unit 740. The one or more demodulated signals from the data buffer 906 that is associated with the one or more detected or monitored analyte levels in one embodiment may be provided to the processing unit 903 for wireless transmission to the receiver unit 710 via the RF transmitter 904 and the antenna 905.

Referring still to FIG. 9, in one embodiment, the processing unit 903 may be configured to send control signals to the output driver that is operatively coupled to the inductive antenna 909 of the transmitter unit 720. In this manner, the processing unit 903 may be configured to control the magnetic field generated by the inductive antenna 909 based on, for example, the detected impedance change of the inductive antenna 806 on the implanted sensor unit 740.

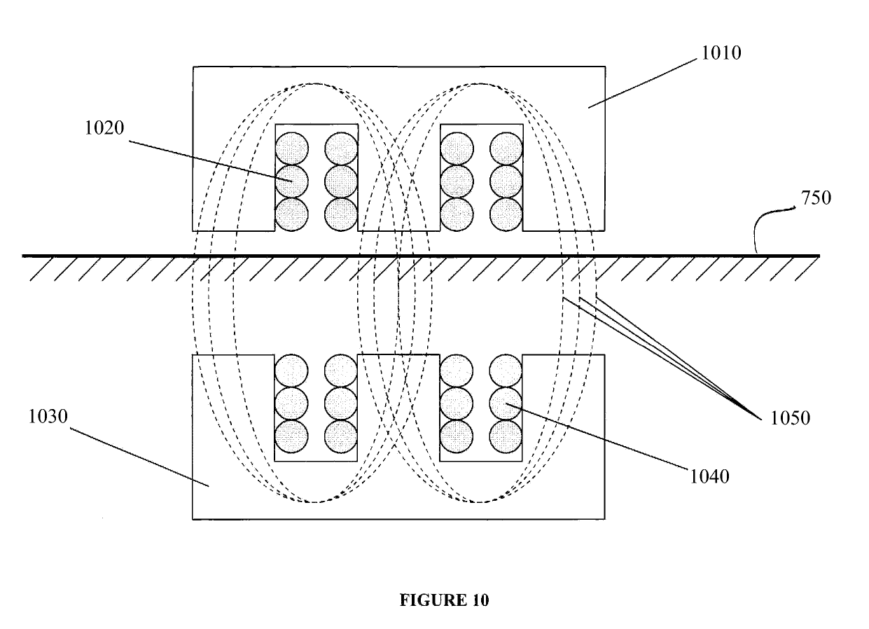

FIG. 10 illustrates the magnetic field generated between the implanted sensor unit and the on-body transmitter unit in accordance with one embodiment of the present invention. Referring to FIG. 10, in one embodiment, a magnetic field 1050 is generated between the inductive antenna 806 of the implanted sensor unit 740 and the inductive antenna 909 of the transmitter unit 720. More specifically, inductive antenna 909 of the transmitter unit in one embodiment includes a pot type ferrite core 1010 and a coil winding 1020, for example, disposed therewith. Further, the inductive antenna 806 of the implanted sensor unit 740 in one embodiment includes a pot type ferrite core 1030 and a coil winding 1040, for example, disposed therewith. In this manner, in one embodiment, the magnetic field 1060 may be generated between the two inductive antennas 806, 909.

FIG. 11 illustrates a pot type ferrite core of the inductive antenna in accordance with one embodiment of the present invention. It is intended that the configuration of the pot type ferrite core shown in FIG. 11 is an exemplary embodiment, and within the scope of the present disclosure, other suitable configurations for the ferrite core may be used.

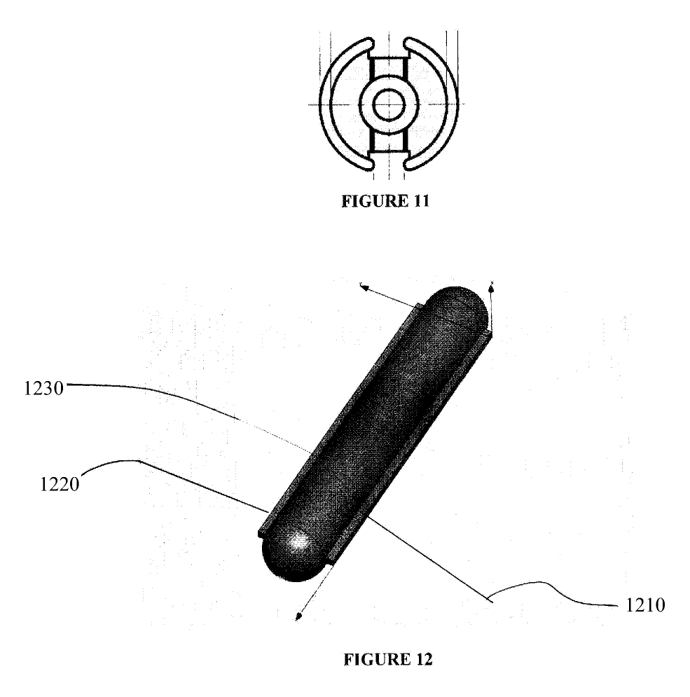

FIG. 12 illustrates the implanted sensor unit in accordance with one embodiment of the present invention. Referring to FIG. 12, the implanted sensor unit 740 in one embodiment may be provided with a housing 1230 and a plurality of guide segments 1210, 1220. In one embodiment, the guide segments 1210, 1220 may be configured to facilitate the positioning of the implanted sensor unit during transcutaneous deployment of the implanted sensor unit so as to accurately position the implanted sensor unit 740 under the skin layer of the patient.

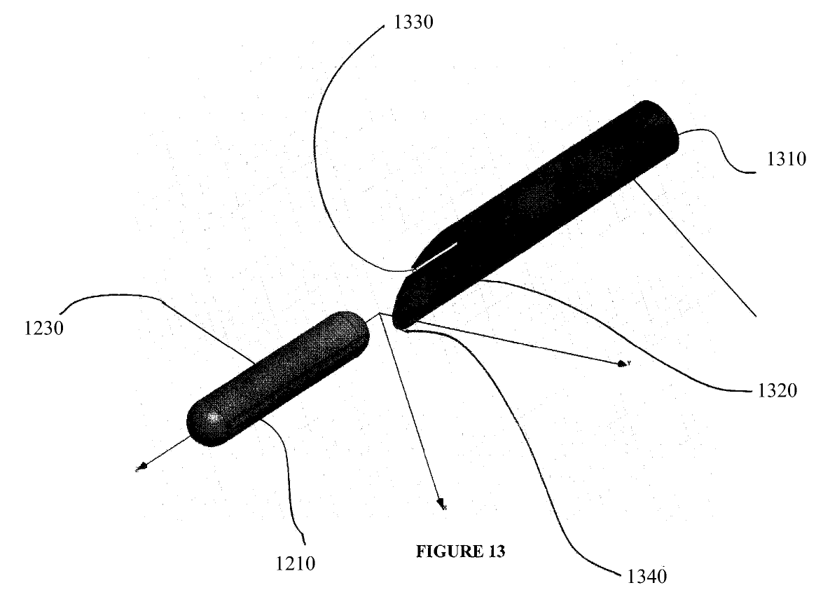

FIG. 13 illustrates an insertion device for use in the transcutaneous implantation of the implanted sensor unit in accordance with one embodiment of the present invention. Referring to FIG. 13, in one embodiment, a tip portion 1310 of the insertion device may include a substantially hollow or tubular opening configured to receive the housing 1230 of the implanted sensor unit 740. The housing 1230 may in one embodiment includes a hermetically sealed biocompatible housing suitable for implantation in the patient. Furthermore, in one embodiment, the tip portion 1310 of the insertion device may be configured to include a plurality of grooves or slits, each configured to correspondingly mate or receive the respective guide segments 1210, 1220 on the housing 1230 of the implanted sensor unit 740.

In this manner, in one embodiment, during deployment, implanted sensor unit 740 may be configured to substantially and securely retained within the tip portion 1310 of the insertion device, and thereafter, to releasably decouple from the tip portion 1310 of the insertion device so as to remain in fluid contact with the patient's analytes at the desired implantation site. Moreover, referring again to FIG. 13, the tip portion 1310 may be provided with a sharp edge 1340 in a beveled tip configuration. The sharp edge 1349 may be configured to readily pierce through the skin barrier of the patient with ease, and possibly minimizing skin trauma and/or pain associated with the implanted sensor unit deployment. Moreover, within the scope of the present invention, the transcutaneous deployment or positioning of the implanted sensor unit 740 may be performed, manually, semi-manually, or automatically using an insertion device.

In the manner described above, in accordance with the various embodiments of the present invention, there are provided method and system for inductively recharging the power supply such as a rechargeable battery of a transmitter unit 102 in the data monitoring and management system 100 using a high frequency magnetic transformer that is provided on the primary and secondary printed circuit boards 603, 604 respectively. Accordingly, a significant reduction in size may be achieved in the transmitter unit 102 design and configuration which may be worn on the patient's body for an extended period of time. Moreover, since the transmitter unit power supply can be recharged without exposing the internal circuitry for example, using a battery cover to periodically replace the battery therein, the transmitter unit housing may be formed as a sealed enclosure, providing water tight seal.

In addition, within the scope of the present invention, the magnetic field generator may be integrated into a flexible arm cuff type device such that the power supply of the transmitter unit 102 may be recharged without being removed from its operating position on the skin of the patient or user, such that the contact between the electrodes of the sensor unit 101 and the transmitter unit 102 analog front end section may be continuously maintained during the active life cycle of the sensor unit 101.

Moreover, in accordance with particular embodiments, there are provided methods and system for inductively charging an implanted sensor unit the data monitoring system 700 using for example, high frequency magnetic transformer that is provided on the primary and secondary printed circuit boards 603, 604 respectively of the transmitter unit 720. In this manner, a compact, extended usage analyte sensor unit may be provided for use in the data monitoring system which does not require a separate power supply such as a battery.

A system in accordance with one embodiment of the present invention includes a hermetically sealed housing, an analyte sensor coupled to the housing for detecting one or more analyte levels of a patient, a power management section coupled to the housing, the power management unit including a power storage unit configured to store charge when in a predetermined proximity to a magnetic field, an data processing unit configured to generate the magnetic field, the data processing unit further configured to receive the one or more analyte levels, and a data monitoring unit wirelessly coupled to the data processing unit, configured to receive one or more signals associated with the one or more analyte levels.

The housing may be substantially entirely implanted under a skin layer of the patient, and analyte sensor may be in fluid contact with an analyte fluid of the patient.

In one aspect, the power management section may include a capacitor. Moreover, the power management section may include, in one embodiment, an application specific integrated circuit (ASIC) chip.

The data processing unit may include a data transmitter unit configured for on-body placement on the patient, where the data transmitter unit may be positioned at a predetermined distance from the housing, which may include, for example, not more than approximately two centimeters.

In another aspect, the data monitoring unit and the data processing unit may be configured to wirelessly communicate using one or more of an RF communication link, a Bluetooth communication link, an infrared communication link, or an 801.1x communication link.

An antenna may be further provided and operatively coupled to the power management section, where the antenna may be configured to magnetically couple to the data processing unit.

An apparatus in accordance with another embodiment of the present invention a housing, an analyte sensor disposed in the housing for detecting one or more analyte levels of a patient, and a power management section disposed in the housing, the power management unit including a power storage unit configured to store charge when in a predetermined proximity to a magnetic field.

In one aspect, the housing may include a hermetically sealed housing.

The housing in a further aspect may include a ferrite core, and also, one or more coil windings disposed on the ferrite core.

In still a further aspect, an inductive antenna may be disposed in the housing and operatively coupled to the power management section.

The power management section may be configured to maintain a predetermined power level in accordance with the generated magnetic field.

A system in accordance with still another embodiment may include an implanted biosensor configured for implantation in a body of a patient, the biosensor configured to detect an analyte level of the patient, an on-body data transmitter magnetically coupled to the implanted biosensor and configured to receive a signal associated with the detected analyte level, and a remote receiver unit configured to wirelessly receive data from the on-body data transmitter.

The implanted biosensor may be substantially entirely implanted in the body of the patient such that the on-body data transmitter does not physically couple to the implanted biosensor.

The implanted biosensor may include an analyte sensor which may include, in one embodiment, a glucose sensor.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

1. A system, comprising:
a hermetically sealed housing;
an analyte sensor coupled to the housing for detecting one or more analyte levels of a patient;
a data processing unit configured to generate a magnetic field, the data processing unit further configured to receive the one or more analyte levels;
a power management section coupled to the housing, the power management unit including a power storage unit configured to store charge when in a predetermined proximity to the magnetic field generated between the housing and the data processing unit external to the housing having a magnetic field strength exceeding a predetermined level, and further, wherein the power management section is configured to draw charge from the power storage unit when the magnetic field strength falls below the predetermined level; and
a data monitoring unit wirelessly coupled to the data processing unit, configured to receive one or more signals associated with the one or more analyte levels.

2. The system of claim 1 wherein the housing is adapted to be substantially entirely implanted under a skin layer of the patient.

3. The system of claim 1 wherein the analyte sensor is adapted to be in fluid contact with an analyte fluid of the patient.

4. The system of claim 1 wherein the power management section includes a capacitor.

5. The system of claim 1 wherein the power management section includes an application specific integrated circuit (ASIC) chip.

6. The system of claim 1 wherein the data processing unit includes a data transmitter unit configured for on-body placement on the patient.

7. The system of claim 6 wherein the data transmitter unit is positioned at a predetermined distance from the housing.

8. The system of claim 7 wherein the predetermined distance is not more than approximately two centimeters.

9. The system of claim 1 wherein the data monitoring unit and the data processing unit are configured to wirelessly communicate using one or more of an RF communication link, an infrared communication link, or an 801.1x communication link.

10. The system of claim 1 further including an antenna operatively coupled to the power management section, and configured to magnetically couple to the data processing unit.

11. An apparatus, comprising:
a housing;
an analyte sensor disposed in the housing for detecting one or more analyte levels of a patient; and
a power management section disposed in the housing, the power management section including a power storage unit configured to store charge when in a predetermined proximity to a magnetic field generated between the housing and a transmitter unit external to the housing having a magnetic field strength exceeding a predetermined level, and further, wherein the power management section is configured to draw charge from the power storage unit when the magnetic field strength falls below the predetermined level.

12. The apparatus of claim 11 wherein the housing includes a hermetically sealed housing.

13. The apparatus of claim 11 wherein the housing includes a ferrite core.

14. The apparatus of claim 13 further including one or more coil windings disposed on the ferrite core.

15. The apparatus of claim 11 further including an inductive antenna disposed in the housing and operatively coupled to the power management section.

16. The apparatus of claim 11 wherein the power management section is configured to maintain a predetermined power level in accordance with the generated magnetic field.

17. A system, comprising:
a biosensor adapted for implantation in a body of a patient, the biosensor configured to detect an analyte level of the patient, the biosensor including a housing and disposed therein a power management section, the power management section including a power storage unit configured to store charge when in a predetermined proximity to a magnetic field having a magnetic field strength exceeding a predetermined level, and further, wherein the power management section is configured to draw charge from the power storage unit when the magnetic field strength falls below the predetermined level;
a data transmitter adapted for positioning on the body of the patient, the data transmitter configured to generate the magnetic field between the data transmitter and the biosensor housing to magnetically coupled to the biosensor and configured to receive a signal associated with the detected analyte level; and
a remote receiver unit configured to wirelessly receive data from the data transmitter.

18. The system of claim 17 wherein the biosensor is adapted to be substantially entirely implanted in the body of the patient such that the data transmitter does not physically couple to the biosensor.

19. The system of claim 17 wherein the biosensor includes an analyte sensor.

20. The system of claim 19 wherein the analyte sensor includes a glucose sensor.
